(19) United States
(12) Patent Application Publication
(10) Pub. No.: US 2022/0233493 A1
(43) Pub. Date: Jul. 28, 2022

(54) SYSTEM AND METHOD FOR CANNABINOID OIL EMULSIFICATION

(71) Applicants: JAMES WOODROW BANNISTER, MOORE, OK (US); DANNY RAY BANNISTER, BEAUMONT, TX (US)

(72) Inventors: JAMES WOODROW BANNISTER, MOORE, OK (US); DANNY RAY BANNISTER, BEAUMONT, TX (US)

(21) Appl. No.: 17/580,718

(22) Filed: Jan. 21, 2022

Related U.S. Application Data
(60) Provisional application No. 63/140,420, filed on Jan. 22, 2021.

(51) Int. Cl. A61K 31/352; A61K 9/107; A61K 31/05; A61K 47/12; A61K 47/26; A61K 47/36; A61K 47/24

(52) U.S. Cl. CPC A61K 31/352; A61K 9/107; A61K 31/05; A61K 47/24; A61K 47/26; A61K 47/36; A61K 47/12

(57) ABSTRACT

Disclosed herein is a system and method for emulsifying cannabinoid oil. According to one embodiment the method comprises mixing cannabinoid oil and a surfactant; heating the distillate; heating distilled water; heating the surfactant; mixing oil with the water; starting the first of two consecutive runs through a high pressure homogenizer system; adding a gumming agent; blending the new mixture; adding lactic acid compound; adding glycerin; blending again; letting the mixture stand; and blending again, thereby producing emulsified cannabinoid oil.

### CROSS REFERENCE TO RELATED APPLICATIONS

This application claims the benefit of U.S. Provisional Patent Application Ser. No. 63/140,420 filed on Jan. 22, 2021, and incorporates said provisional application, including its appendix and figures, by reference into this document as if fully set out at this point.

### TECHNICAL FIELD

This disclosure relates generally to systems and methods of emulsification of oil obtained from cannabis or hemp plants.

### BACKGROUND

Cannabinoid oil (e.g., cannabidiol) is rapidly gaining in popularity as acceptance by the public broadens and states move to legalize medical and recreational use of marijuana. It is extracted from cannabis and hemp and is widely recognized as having health benefits. It is appearing with increasing frequency in prescription and nonprescription food, beverages, topical cremes, tinctures, capsules, lotions, etc. Cannabinoids are not soluble in water, but they show good solubility in non-polar solvents such as oils and fats. The hydrophobic nature of cannabinoid oil makes incorporating it into such consumer packaged goods products problematic and this is especially true in the case of products that are water based.

One compound of particular importance that is found in cannabinoid oil is CBD. Since CBD does not mix well with water, it is conventional to try to emulsify it to create very small protected droplets (a nanoemulsion) that will remain dispersed in an aqueous solution. The goal of such an approach is to prevent the droplets from rejoining and separating from the water solution again to form an oil layer on top of the product. When emulsification is successful the oil and water combination remain uniformly and equally dispersed throughout the surrounding liquid during manufacture, transportation, and eventual use.

Emulsion of the CBD results in even dispersion throughout the product, which means that consumers will get the same amount of an active ingredient (CBD or THC) in each use. This avoids scenarios where there is a high concentration layer of CBD floating on the top of the product which is used before the material it was dispersed it. Similarly, this helps ensure that when the bottom of the container is reached the last few portions will have the same amount of CBD in them as the first. Thus, product quality can be maintained throughout.

Macroemulsions are typically defined as emulsions which have droplets that are 1 micron in diameter. These emulsions typically lack shelf stability due to their low solubility and tendency to separate into water land oil layers, making them unsuitable for most water-based products or beverages. On the other hand, microemulsions feature droplet diameters below 100 nanometers. These emulsions tend to be more stable and are preferred for uses that involve water-based products.

However, the emulsion process is a complex one that can often in an inferior mixture that is not shelf stable. This what is needed is a method of emulsification of CBD which does not suffer from the disadvantages of the prior art.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### SUMMARY OF THE INVENTION

Disclosed herein is a system and method for emulsifying cannabinoid oil, including compounds like CBD and THC that are found therein. One embodiment comprises the steps of using a mixture ratio of between 1 to 4 parts cannabinoid oil to 1 part surfactant; heating the distillate to between about 170° F. and 195° F.; heating distilled water to at least 212° F.; heating the surfactant to between about 110° F. and 125° F.; mixing the CBD oil with between 1-4 parts water while the temperature is between 145 F and 215° F. then mixing at a rate of between 1500 to 6200 rpm for at least 3 minutes; while mixture is between 145° F. and 215° F. and within 3 minutes of mixing, starting the first of two consecutive runs through a high pressure homogenizer system. Start running within 3 minutes of mixing; adding between 3 g and 6 g of gumming agent per 1000 mL of the mixture, within 5 minutes after the second run through the high pressure homogenizer system; blending new mixture at between 1500 and 3500 rpm for between 2 and 5 minutes; adding between 15 and 35 ml of lactic acid compound per 1000 ml of mixture; adding between 25 and 75 mL of glycerin per 1000 mL; blending again at between 1500 and 3500 rpm; letting mixture stand for four hours; and blending again at between 1500 and 3500 rpm, thereby producing said emulsified cannabinoid oil.

The foregoing has outlined in broad terms some of the more important features of the invention disclosed herein so that the detailed description that follows may be more clearly understood, and so that the contribution of the instant inventors to the art may be better appreciated. The instant invention is not to be limited in its application to the details of the construction and to the arrangements of the components set forth in the following description or illustrated in the drawings. Rather, the invention is capable of other embodiments and of being practiced and carried out in various other ways not specifically enumerated herein. Finally, it should be understood that the phraseology and terminology employed herein are for the purpose of description and should not be regarded as limiting, unless the specification specifically so limits the invention.

### BRIEF DESCRIPTION OF THE DRAWINGS

These and further aspects of the invention are described in detail in the following examples and accompanying drawings.

FIG. 1 contains a schematic illustration of an embodiment of the invention.

### DETAILED DESCRIPTION

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

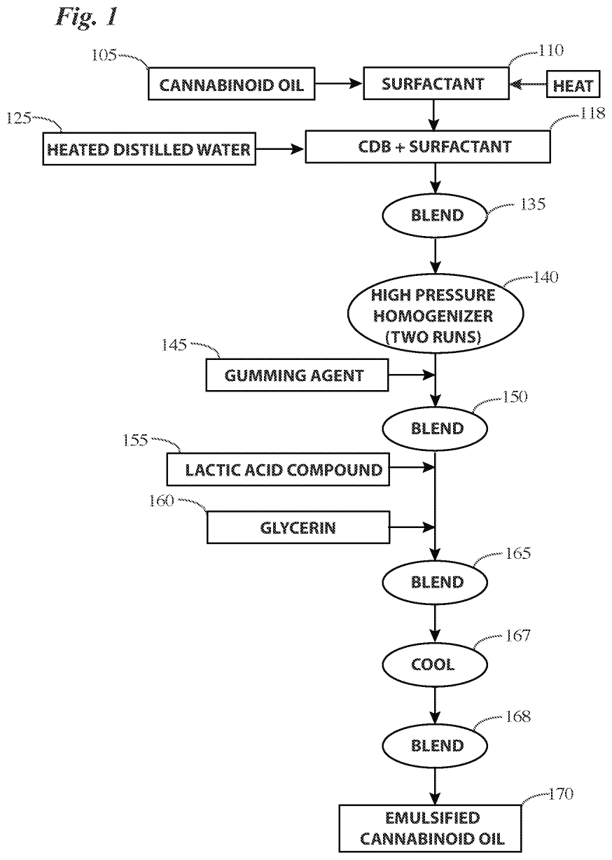

Referring to FIG. 1, this FIGURE contains one possible processing sequence suitable for use with an embodiment.
Heat surfactant 110 to a temperature of between about 110° F. and 125° F. In some embodiments the surfactant will be phosphatidylcholine. Other surfactants that might be acceptable in some circumstances include phospholipids such as phosphatidylethanolamine, phosphatidylcholine and others. Note that one important role of the surfactant is to create liposomes that surround the cannabinoid oil that is added in the next step.
Add about 1 to 4 parts cannabinoid oil 105 to 1 part of the heated surfactant 110 and mix to produce a cannabinoid/surfactant mixture. In some embodiments, a mixture of 2 parts cannabinoid oil to 1 part heated surfactant yields good results. Heat the cannabinoid distillate mixture to a temperature of between about 170° F. and 195° F.
To the heated cannabinoid distillate mixture, add about 1 to 4 parts hot distilled water 125 to one part of the heated cannabinoid distillate mixture and mix. Preferably the temperature of the distilled water will be at least about 212° F.
While the temperature of the mixture 118 is between about 145° F. and 215° F., blend it at a rate of between 1500 to 6200 rpm for at least 1-5 minutes (step 135). The temperature after the previous step will likely be within the desired range. However, if the temperature falls below 145° the mixture may need to be heated. So, for purposes of the instant disclosure the phrase “securing a temperature” will be interpreted to mean proceeding if the temperature is within the stated range or heating the mixture if its temperature is below the preferred range. Note that in this embodiment the length of time that the mixture will need to be blended depends, among other things, on the thickness of the mixed product. For example, a thicker mixture will likely require additional blending time. A person of ordinary skill in the art will recognize that if the mixture is too thick it will not work well in the step that follows and will understand how such might be adjusted by, for example, further dilution with water and additional mixing.
While the blended mixture is between about 145° F. and 215° F., start the first of two runs through a high-pressure homogenizer system 140. The first run should be started running within about 3 minutes of blending (step 135). The second run should follow promptly after the first run. Note that in some instances one run through the high-pressure homogenizer system might be enough. However, generally speaking two or three runs will usually be necessary.
Add between 3 g and 6 g of gumming agent 145 per 1000 mL of the homogenized mixture. Preferably the gumming agent will be added within about 5 minutes after the run through the high-pressure homogenizer system. In some embodiments, the gumming agent will be Xanthan gum. Adding the gumming agent is an important step in the instant process.
Blend (step 150) the homogenized mixture plus gumming agent at between about 1500 and 3500 rpm for between about 2 and 5 minutes. That being said, those of ordinary skill in the art will recognize that a longer or shorter blending time might be necessary.
As a next optional step, add between about 15 and 35 ml of lactic acid compound per 1000 ml of blended mixture from the previous step. In one embodiment, the lactic acid compound will be Purac™. This is a brand name of a lactic acid preservative. This particular product is made from fermented cane sugars. Potassium sorbate is an example of another preservative that might be used, although the lactic acid compound is usually preferred. Note that this is done to increase the shelf life of the final product and, as such, it should be considered an optional step in connection with emulsifying the CBD oil.
Add between about 25 and 75 mL of glycerin per 1000 mL (step 160). Note that this step is optional but generally preferable. One function of the glycerin is that it tends to smooth out the gumming agent which, in some variations, can have a somewhat granular consistency when added to the mixture. Depending on the circumstances and choice of gumming agent, the addition of glycerin may not be strictly necessary.
Blend again (step 165) at between about 1500 and 3500 rpm. This step may not be necessary if glycerin is not added.
Let the mixture stand and cool for a period of time (step 167). This step allows the gumming agent time to create a homogeneous solution and absorb excess water. One to four hours will usually be a satisfactory cooling period. Obviously, the time required for this step might be longer or shorter depending on the initial temperature of the mixture and other factors. Those of ordinary skill in the art will readily be able to choose an appropriate cooling period in order to allow the mixture to reach an acceptable degree of consistency.
Blend (step 168) the now homogenized mixture from the previous mixture again at between about 1500 and 3500 rpm for about one to five minutes or until mixture is smooth to produce as a final product emulsified cannabinoid oil 170.

It should be noted and remembered that cannabinoids comprise a family of over 100 compounds including, without limitation, CBD, CBC, CBG, CBN, etc., as well as the THC family of compounds including, without limitation, THC Delta 8, Delta 9, Delta 10, etc.

Although the instant disclosure uses CBD as an example compound, that was provided to illustrate the general steps associated with an embodiment and was not intended to limit the scope of the claims that follow. The methods discussed herein would be applicable to any of the family of compounds found in cannabinoid oil, including mixtures thereof.

While this invention is susceptible of embodiment in many different forms, there is shown in the drawings, and will herein be described hereinafter in detail, some specific embodiments of the instant invention. It should be understood, however, that the present disclosure is to be considered an exemplification of the principles of the invention and is not intended to limit the invention to the specific embodiments or algorithms so described.

It is to be understood that the terms “including”, “comprising”, “consisting” and grammatical variants thereof do not preclude the addition of one or more components, features, steps, or integers or groups thereof and that the terms are to be construed as specifying components, features, steps, or integers.

If the specification or claims refer to “an additional” element, that does not preclude there being more than one of the additional element.

It is to be understood that where the claims or specification refer to “a” or “an” element, such reference is not to be construed that there is only one of that element.

It is to be understood that where the specification states that a component, feature, structure, or characteristic “may”, “might”, “can” or “could” be included, that particular component, feature, structure, or characteristic is not required to be included.

Where applicable, although state diagrams, flow diagrams or both may be used to describe embodiments, the invention is not limited to those diagrams or to the corresponding descriptions. For example, flow need not move through each illustrated box or state, or in exactly the same order as illustrated and described.

Methods of the present invention may be implemented by performing or completing manually, automatically, or a combination thereof, selected steps or tasks.

The term “method” may refer to manners, means, techniques and procedures for accomplishing a given task including, but not limited to, those manners, means, techniques and procedures either known to, or readily developed from known manners, means, techniques and procedures by practitioners of the art to which the invention belongs.

For purposes of the instant disclosure, the term “at least” followed by a number is used herein to denote the start of a range beginning with that number (which may be a ranger having an upper limit or no upper limit, depending on the variable being defined). For example, “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. Terms of approximation (e.g., “about”, “substantially”, “approximately”, etc.) should be interpreted according to their ordinary and customary meanings as used in the associated art unless indicated otherwise. Absent a specific definition and absent ordinary and customary usage in the associated art, such terms should be interpreted to be ±10% of the base value.

When, in this document, a range is given as “(a first number) to (a second number)” or “(a first number)-(a second number)”, this means a range whose lower limit is the first number and whose upper limit is the second number. For example, 25 to 100 should be interpreted to mean a range whose lower limit is 25 and whose upper limit is 100. Additionally, it should be noted that where a range is given, every possible subrange or interval within that range is also specifically intended unless the context indicates to the contrary. For example, if the specification indicates a range of 25 to 100 such range is also intended to include subranges such as 26-100, 27-100, etc., 25-99, 25-98, etc., as well as any other possible combination of lower and upper values within the stated range, e.g., 33-47, 60-97, 41-45, 28-96, etc. Note that integer range values have been used in this paragraph for purposes of illustration only and decimal and fractional values (e.g., 46.7-91.3) should also be understood to be intended as possible subrange endpoints unless specifically excluded.

It should be noted that where reference is made herein to a method comprising two or more defined steps, the defined steps can be carried out in any order or simultaneously (except where context excludes that possibility), and the method can also include one or more other steps which are carried out before any of the defined steps, between two of the defined steps, or after all of the defined steps (except where context excludes that possibility).

Further, it should be noted that terms of approximation (e.g., “about”, “substantially”, “approximately”, etc.) are to be interpreted according to their ordinary and customary meanings as used in the associated art unless indicated otherwise herein. Absent a specific definition within this disclosure, and absent ordinary and customary usage in the associated art, such terms should be interpreted to be plus or minus 10% of the base value.

Still further, additional aspects of the instant invention may be found in one or more appendices attached hereto and/or filed herewith, the disclosures of which are incorporated herein by reference as if fully set out at this point.

Thus, the present invention is well adapted to carry out the objects and attain the ends and advantages mentioned above as well as those inherent therein. While the inventive device has been described and illustrated herein by reference to certain preferred embodiments in relation to the drawings attached thereto, various changes and further modifications, apart from those shown or suggested herein, may be made therein by those of ordinary skill in the art, without departing from the spirit of the inventive concept the scope of which is to be determined by the following claims.

What is claimed is:

1. A method of producing an emulsified cannabinoid oil, comprising the steps of:
(a) heating a surfactant to a temperature between 110° F. and 125° F.;
(b) adding to said surfactant between 1 to 4 parts of cannabinoid oil for each part of surfactant to produce a cannabinoid/surfactant mixture;
(c) heating said cannabinoid/surfactant mixture to a temperature between 170° F. and 195° F.;
(d) adding to said cannabinoid/surfactant mixture a quantity of hot distilled water in a ratio of between 1 to 4 parts hot distilled water to 1 part of said cannabinoid/surfactant mixture, thereby producing a diluted mixture;
(e) securing a temperature of said diluted mixture between 145° F. and 215° F. and blending said diluted mixture at a rate of between 1500 and 6200 RPM to produce a blended heated mixture;
(f) while said blended heated mixture has a temperature of between 145° F. and 215° F., passing said blended heated mixture through a high-pressure homogenizer system at least one time, thereby producing a homogenized mixture;
(g) adding to said homogenized mixture between 3 g and 6 g of a gumming agent per 1000 ml of said homogenized mixture;
(h) allowing said homogenized mixture to cool for a period of time; and
(i) blending said cooled homogenized mixture at a rate of between 1500 and 3500 RPM until said cooled homogenized mixture has a smooth consistency, thereby producing said emulsified cannabinoid oil.

2. The method of claim 1 wherein the cannabinoid oil comprises at least one of CBD, CBC, CBG, CBN, THC Delta 8, Delta 9, and Delta 10.

3. The method of claim 1 wherein the cannabinoid oil comprises a THC compound.

4. The method of claim 1, wherein said gumming agent is an Xanthan gum.

5. The method of claim 1, wherein step (f) comprises the steps of:
(f1) adding to said homogenized mixture between 3 g and 6 g of said gumming agent per 1000 ml of said homogenized mixture; and
(f2) adding a quantity of a preservative to said homogenized mixture.

6. The method of claim 5, wherein said preservative is a lactic acid preservative.

7. The method of claim 5, wherein said preservative is potassium sorbate.

8. The method of claim 5, wherein said gumming agent is an Xanthan gum.

9. The method of claim 1, wherein said blended heated mixture is passed through a high-pressure homogenizer system two or three times, thereby producing said homogenized mixture.

10. The method of claim 1, wherein said surfactant is phosphatidylcholine.

11. The method of claim 1, wherein step (h) comprises the step of allowing said homogenized mixture to cool for between one and four hours.

12. The method of claim 1, wherein step (i) comprises the step of blending said cooled homogenized mixture at a rate of between 1500 and 3500 RPM for one to five minutes, thereby producing said emulsified cannabinoid oil.

13. A method of producing an emulsified cannabinoid oil, comprising the steps of:
(a) heating a surfactant to a temperature between 110° F. and 125° F.;
(b) adding 1 to 4 parts of cannabinoid oil to said heated surfactant for each part of said heated surfactant to produce a cannabinoid/surfactant mixture;
(c) adding 1 to 4 parts hot distilled water for each 1 part of said cannabinoid/surfactant mixture, thereby producing a heated mixture;
(d) while a temperature of said heated mixture is between 145° F. and 215° F., blending said heated mixture at a rate of between 1500 RPM and 6200 RPM for at least 1 to 5 minutes to produce a blended heated mixture;
(e) while a temperature of said blended heated mixture is between 145° F. and 215° F., passing said blended heated mixture through a high-pressure homogenizer system at least one time, thereby producing a homogenized mixture;
(f) adding to said homogenized mixture between 3 g and 6 g of a gumming agent per 1000 ml of said homogenized mixture and blending said gumming agent and said homogenized mixture at a rate of between 1500 RPM and 3500 RPM for 2 to 5 minutes, thereby producing a blended homogenized mixture;
(g) adding to said blended homogenized mixture between 15 ml and 35 ml of lactic acid compound per 1000 ml of blended homogenized mixture;
(h) adding to said blended homogenized mixture between 25 ml and 75 ml of glycerin per 1000 ml of said blended homogenized mixture;
(i) blending said blended homogenized mixture containing said glycerin and said lactic acid compound at a rate of between 1500 RPM and 3500 RPM to produce a blended mixture;
(j) allowing said blended mixture to cool for one to four hours; and
(k) blending said blended mixture at a rate of between 1500 and 3500 RPM for one to five minutes, thereby producing said emulsified cannabinoid oil.

14. The method of claim 13 wherein the cannabinoid oil comprises at least one of CBD, CBC, CBG, CBN, THC Delta 8, Delta 9, and Delta 10.

15. The method of claim 13 wherein the cannabinoid oil comprises a THC compound.

16. The method of claim 13, wherein said gumming agent is an Xanthan gum.

17. The method of claim 13, wherein said surfactant is phosphatidylcholine.
